(19) United States
(12) Patent Application Publication
(10) Pub. No.: US 2026/0013809 A1
(43) Pub. Date: Jan. 15, 2026

(54) X-RAY CT APPARATUS, DISPLAY METHOD, AND NON-TRANSITORY COMPUTER READABLE MEDIUM

(71) Applicants: KEIO UNIVERSITY, Tokyo (JP); Canon Medical Systems Corporation, Otawara-shi (JP)

(72) Inventors: Minoru YAMADA, Hino (JP); Masahiro JINZAKI, Tokyo (JP); Yoshitake YAMADA, Tokyo (JP); Yoichi YOKOYAMA, Tokyo (JP); Jun OOSHIMA, Nasushiobara (JP)

(73) Assignees: KEIO UNIVERSITY, Tokyo (JP); Canon Medical Systems Corporation, Otawara-shi (JP)

(21) Appl. No.: 19/263,705

(22) Filed: Jul. 9, 2025

(30) Foreign Application Priority Data
Jul. 12, 2024 (JP) 2024-112691

(51) Int. Cl. A61B 6/03; A61B 6/04; A61B 6/08; A61B 6/46

(52) U.S. Cl. CPC A61B 6/035; A61B 6/0492; A61B 6/08; A61B 6/462

(57) ABSTRACT

According to one embodiment, an X-ray CT apparatus includes a scanner, a stand, and a processing circuit. The scanner has an opening into which a subject is inserted. The stand supports the scanner so as to enable the scanner to move in a vertical direction. The processing circuit acquires an inner image captured by imaging an inside of the opening from a camera. The processing circuit generates a visualized image by visualizing the inside of the opening viewed from a visual line direction toward the inside of the opening based on the inner image. The processing circuit causes a display unit to display the visualized image.

### CROSS-REFERENCE TO RELATED APPLICATIONS

This application is based upon and claims the benefit of priority from Japanese Patent Application No. 2024-112691, filed Jul. 12, 2024, the entire contents of which are incorporated herein by reference.

### FIELD

Embodiments described herein relate generally to an X-ray CT apparatus, a display method, and a non-transitory computer readable medium.

### BACKGROUND

An X-ray CT (Computed Tomography) apparatus can image a subject in various positions. The X-ray CT apparatus can image a subject in a standing position or sitting position by moving the scanner installed horizontally in the vertical direction.

Conventionally, an upright X-ray CT apparatus projects laser light from the projector in accordance with the height of an imaging section (or X-ray path) inside the opening of the scanner. An examination technician checks the laser light projected on a subject and aligns the height of an imaging section with a desired height in a subject.

The examination technician, however, needs to check the laser light projected on the subject from below the opening. The examination technician looks into the opening while kneeling down (taking a semi-crouching position) and hence experiences a large physical load.

### BRIEF DESCRIPTION OF THE DRAWINGS

FIG. 1 is a schematic view showing the overall arrangement of an X-ray CT apparatus according to the first embodiment.

FIG. 2 is a top view showing the arrangement of a gantry according to the first embodiment.

FIG. 3 is a flowchart showing the operation of the X-ray CT apparatus according to the first embodiment.

FIG. 4 is an explanatory diagram showing a display example of a visualized image according to the first embodiment.

FIG. 5 is a top view showing the arrangement of a gantry according to the second embodiment.

FIG. 6 shows explanatory diagrams showing a display example of a visualized image according to the second embodiment.

FIG. 7 is a top view showing the arrangement of a gantry according to the third embodiment.

FIG. 8 is a top view showing the arrangement of a gantry according to the fourth embodiment.

FIG. 9 shows top views showing the arrangement of a gantry according to the fifth embodiment.

FIG. 10 is a flowchart showing the operation of an X-ray CT apparatus according to the fifth embodiment.

FIG. 11 shows top views showing the arrangement of a gantry according to the sixth embodiment.

FIG. 12 is a flowchart showing the operation of an X-ray CT apparatus according to the sixth embodiment.

FIG. 13 shows explanatory diagrams showing a display example of a visualized image according to the sixth embodiment.

### DETAILED DESCRIPTION

In general, according to one embodiment, an X-ray CT apparatus includes a scanner, a stand, and a processing circuit. The scanner has an opening into which a subject is inserted. The stand supports the scanner so as to enable the scanner to move in a vertical direction. The processing circuit acquires an inner image captured by imaging an inside of the opening from a camera. The processing circuit generates a visualized image by visualizing the inside of the opening viewed from a visual line direction toward the inside of the opening based on the inner image. The processing circuit causes a display unit to display the visualized image. Each embodiment will be described below with reference to the accompanying drawings. A plurality of parts denoted by the same reference numerals are regarded as identical, and a repetitive description of them will be omitted.

### First Embodiment

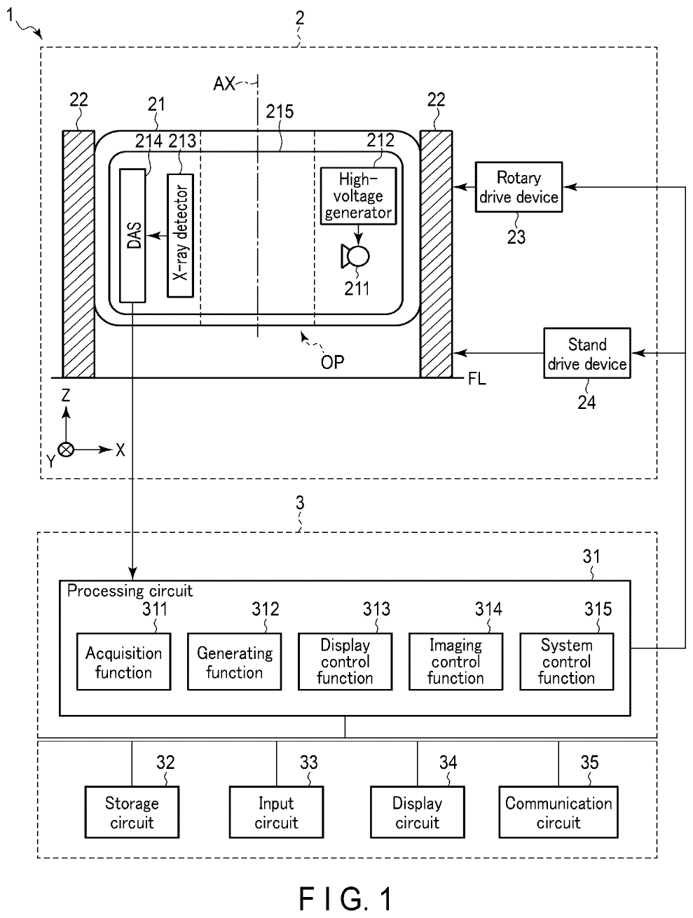

FIG. 1 is a schematic view showing the overall arrangement of an X-ray CT apparatus 1 according to the first embodiment. The X-ray CT apparatus 1 irradiates a subject with X-rays from an X-ray tube and detects the applied X-rays with an X-ray detector. The X-ray CT apparatus 1 generates a CT image concerning the subject based on an output from the X-ray detector.

The X-ray CT apparatus 1 includes a gantry 2 and a console 3. For example, the gantry 2 is installed in an examination room, and the console 3 is installed in an operation room adjacent to the examination room. The gantry 2 and the console 3 are communicably connected to each other wiredly or wirelessly. The gantry 2 has a mechanism for performing X-ray computed tomography (to be referred to as CT imaging hereinafter) of a subject in a lying position, standing position, or sitting position. The console 3 is a computer that controls the gantry 2.

(Arrangement of Gantry) The gantry 2 includes a scanner 21 and two stands 22. The scanner 21 performs X-ray CT imaging. The scanner 21 is a substantially cylindrical structure having an opening OP. The opening OP is also called a “bore”.

A three-dimensional orthogonal coordinate system is defined with respect to the gantry 2. The orthogonal coordinate system has an X-axis, a Y-axis, and a Z-axis which are perpendicular to each other. The X-axis direction is the direction (first horizontal direction) passing through the two stands 22 so as to be parallel to a floor surface FL. The Y-axis direction is a direction (second horizontal direction) parallel to the floor surface FL and perpendicular to the X-axis. The Z-axis direction is the direction (vertical direction) perpendicular to the floor surface FL.

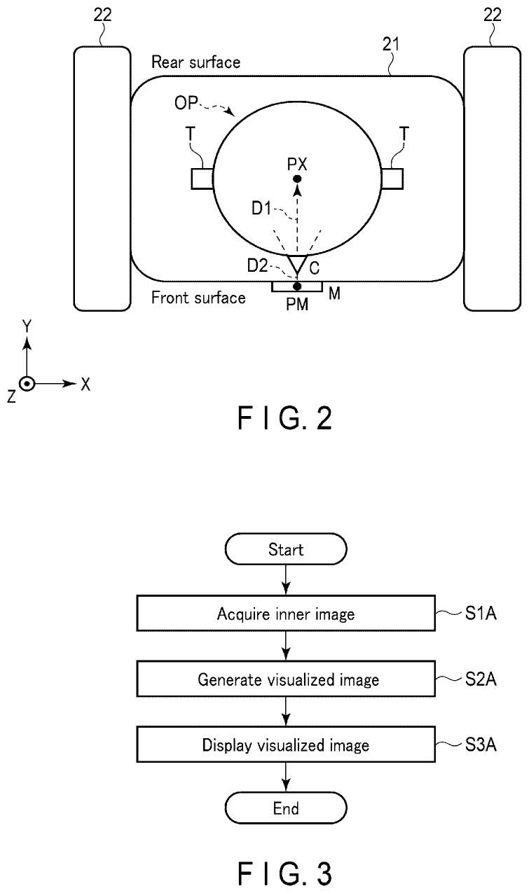

FIG. 2 is a top view showing the arrangement of the gantry 2 according to the first embodiment. FIG. 2 shows the scanner 21 and the two stands 22 viewed from the positive Z-axis direction (that is, from above). The scanner 21 has six surfaces (an upper surface, a lower surface, a front surface, a rear surface, a right-side surface, and a left-side surface). The two surfaces (upper and lower surfaces) are also called “bottom surfaces”. The four surfaces (front, rear, right-side, and left-side surfaces) are also called “side surfaces”. The “front surface” is a surface facing the abdominal side of a subject. The “rear surface” is a surface facing the dorsal side of the subject. The “right-side surface” is a surface on the right side when the abdominal side of the subject is viewed from the outside of the scanner 21. The “left-side surface” is a surface on the left side when the abdominal side of the subject is viewed from the outside of the scanner 21. Each surface of the stand 22 is defined in the same manner as each surface of the scanner 21.

The scanner 21 includes a camera C on a circular inner peripheral side surface forming the opening OP. For example, the camera C is installed so as to face a central position PX of the opening OP from the front surface of the scanner 21. The camera C captures an optical image (that is, an inner image) of the inside of the opening OP from a first visual line direction D1. The camera C transmits the captured inner image to the console 3.

The camera C may be a digital camera or infrared camera. The camera C may be installed at an arbitrary position where the inside of the opening OP can be imaged. For example, the camera C is installed on the ceiling or wall surface of the examining room where the gantry 2 is installed. The camera C may be installed on the rotating frame (that is, the rotating portion) or the main frame (that is, the fixing portion) that supports the rotating frame.

The first visual line direction D1 is the direction defined by the installation position of the camera C as a starting point and the central position PX of the opening OP as an ending point. The first visual line direction D1 is also a normal direction perpendicular to a tangent line passing through the installation position of the camera C. The central position PX is a point (for example, an isocenter) on a central axis AX of the opening OP.

The scanner 21 further includes two projectors T on the inner peripheral side surface forming the opening OP. The two projectors T are installed so as to be symmetrical about the central axis AX (or the central position PX) of the opening OP. Only one of the two projectors T may be installed. Under the control of the console 3, the two projectors T project laser light (for example, infrared light or visible light) for positioning the subject inserted into the opening OP. The projected light indicates the height of an imaging section (or X-ray path). The imaging section may be an end or middle imaging section of a plurality of imaging sections. The color of light projected is arbitrary (for example, red, green, or blue).

A monitor M is installed on the front surface of the scanner 21. The monitor M is installed at a position that bisects the length of the front surface of the scanner 21 in the long axis direction (X-axis direction). The monitor M displays a visualized image when the inner space of the opening OP is viewed from a second visual line direction D2. The second visual line direction D2 is the direction defined by the installation position PM of the monitor M as a starting point and the central position PX of the opening OP as an ending point. In this embodiment, the second visual line direction D2 is on the same straight line as the first visual line direction D1. The monitor M is an example of a display unit.

Refer back to FIG. 1. The scanner 21 includes an X-ray tube 211, a high-voltage generator 212, an X-ray detector 213, and a DAS 214. The X-ray tube 211 and the high-voltage generator 212 are installed so as to face the X-ray detector 213 and the DAS 214 through the opening OP. DAS stands for Data Acquisition System.

More specifically, the scanner 21 includes a main frame (not shown) and a rotating frame 215. The main frame is formed from a metal such as aluminum. The main frame supports the rotating frame 215 so as to allow it to rotate about the central axis AX through a bearing or the like. The contact portion between the main frame and the rotating frame 215 is provided with an annular electrode (not shown) and also provided with a conductive slider (not shown) in slidable contact with the annular electrode. The rotating frame 215 is formed from a metal such as aluminum in an annular shape. For example, the X-ray tube 211 and the X-ray detector 213 are attached to the rotating frame 215.

The rotating frame 215 receives drive power from a rotary drive device 23 and rotates about the central axis AX of the opening OP. The rotary drive device 23 generates drive power for rotating the rotating frame 215 under the control of the console 3. The rotary drive device 23 is implemented by, for example, a motor such as a direct drive motor or servo motor.

The stand 22 is a structure that supports the scanner 21 with a space from the floor surface FL. The stand 22 is also called a “support pillar”. The stand 22 has, for example, a columnar shape such as a cylindrical columnar shape or prismatic columnar shape. The stand 22 is attached to, for example, a side surface of the scanner 21. In order to perform CT imaging of a subject in a standing position or sitting position, the stand 22 supports the scanner 21 so as to allow it to slide in a direction perpendicular to the floor surface FL while being in a position in which the central axis AX of the opening OP is maintained in a direction perpendicular to the floor surface FL. The floor surface FL is an example of a surface on which the stand 22 is mounted. The floor surface FL may be a surface on which the soles of the feet of a subject in a standing state or sitting state are placed or a surface on which a support tool is installed.

Typically, the stands 22 are provided on the both side surfaces (the right-side surface and the left-side surface) of the scanner 21. Alternatively, one stand 22 may be provided on one side surface of the scanner 21. The stand 22 may have a U shape or the like as long as it can support at least one side surface of the scanner 21.

The stand 22 may support the scanner 21 so as to allow it to rotate about a horizontal axis (to be referred to as a tilt axis hereinafter) parallel to the floor surface FL. In this case, the scanner 21 and the stand 22 may be connected to each other through a bearing or the like. This arrangement enables the gantry 2 to execute CT imaging of a subject in a standing position (standing position imaging), CT imaging of a subject in a sitting position (sitting position imaging), and CT imaging of a subject in a lying position (lying position imaging).

The stand 22 may accommodate a stand drive device 24 for allowing the scanner 21 to slide in the vertical direction. The stand drive device 24 generates drive power for causing the scanner 21 to slide in the vertical direction under the control of the console 3. More specifically, the stand drive device 24 generates drive power by driving at a rotating speed corresponding to the duty ratio or the like of a drive signal from the console 3. The stand 22 receives drive power from the stand drive device 24 and causes the scanner 21 to slide in the vertical direction with respect to the stand 22. The stand drive device 24 is implemented by, for example, a motor such as a servo motor.

The X-ray tube 211 generates X-rays upon receiving a high voltage from the high-voltage generator 212. The high-voltage generator 212 is attached to, for example, the rotating frame 215. The high-voltage generator 212 generates a high voltage to be applied to the X-ray tube 211 from the power supplied from the power supply device (not shown) of the scanner 21 through the annular electrode under the control of the console 3. The high-voltage generator 212 and the X-ray tube 211 are connected to each other through a high-voltage cable (not shown). The high voltage generated by the high-voltage generator 212 is applied to the X-ray tube 211 through the high-voltage cable.

The X-ray detector 213 detects the X-rays generated from the X-ray tube 211 and transmitted through a subject. The X-ray detector 213 has a plurality of X-ray detection elements (not shown) arrayed on a two-dimensional curved surface defined by the column direction and the channel direction. The column direction is defined by the Z-axis direction. The channel direction is defined by a direction along an arc orthogonal to the column direction. Each X-ray detection element detects X-rays from the X-ray tube 211 and converts the X-rays into an electrical signal having a crest value corresponding to the intensity of the detected X-rays. Each X-ray detection element includes, for example, a scintillator and a photoelectric conversion element.

The scintillator generates fluorescent light upon receiving X-rays. The photoelectric conversion element converts the generated fluorescent light into an electric charge pulse. The electric charge pulse has a crest value corresponding to the intensity of the X-rays. The photoelectric conversion element may be a circuit element (for example, a photomultiplier or photodiode) that converts fluorescent light into an electrical signal. The X-ray detector 213 may be an indirect conversion type detector that converts X-rays into an electrical signal after converting the X-rays into fluorescent light or a direct conversion type detector that directly converts X-rays into an electrical signal.

The DAS 214 acquires digital data indicating the intensity of X-rays attenuated by a subject for each view. The DAS 214 is connected to, for example, the X-ray detector 213. The DAS 214 includes an integration circuit and an A/D converter. The integration circuit generates an integral signal by integrating electrical signals from the X-ray detection element over a predetermined view period. The A/D converter A/D-converts the integral signal to generate digital data (projection data) having a data value corresponding to the crest value of the integral signal. The projection data is a set of the digital values of X-ray doses identified by column numbers, channel numbers, and view numbers. The projection data is supplied to the console 3 via a noncontact data transmitter (not shown) accommodated in the scanner 21.

The rotary drive device 23 is a device that generates drive power for rotating the rotating frame 215. The rotary drive device 23 generates drive power under the control of the console 3. The rotary drive device 23 supplies the generated drive power to the rotating frame 215. The rotary drive device 23 may tilt the central axis AX of the opening OP around a tilt axis (the X-axis in particular) parallel to the floor surface FL.

The stand drive device 24 is a device that generates drive power for the stand 22. The stand drive device 24 generates drive power under the control of the console 3. The stand drive device 24 supplies the generated drive power to the stand 22. The stand drive device 24 may drive the stand 22 in the horizontal direction (X-axis direction or Y-axis direction).

(Arrangement of Console) The console 3 controls the high-voltage generator 212, the DAS 214, the rotary drive device 23, the stand drive device 24, and the like. The console 3 includes a processor such as a CPU (Central Processing Unit) and storage devices (memories) such as a ROM (Read Only Memory) and a RAM (Random Access Memory) as hardware resources.

The console 3 includes a processing circuit 31, a storage circuit 32, an input circuit 33, a display circuit 34, and a communication circuit 35 as constituent elements. Data communication between the respective constituent elements is performed via a bus (BUS). At least some of the constituent elements may be included in the gantry 2.

The processing circuit 31 is a circuit that comprehensively controls each constituent element of the console 3. The processing circuit 31 has at least one processor. The processor means a circuit such as a CPU, GPU (Graphics Processing Unit), ASIC (Application Specific Integrated Circuit), or PLD (Programmable Logic Device).

The programmable logic device means a circuit such as an SPLD (Simple Programmable Logic Device), CPLD (Complex Programmable Logic Device), or FPGA (Field Programmable Gate Array). The processing circuit 31 is an example of a processing unit.

If the processor is a CPU, the CPU implements each function by reading out and executing a corresponding one of the programs stored in the storage circuit 32. If the processor is an ASIC, each function is incorporated as a logic circuit in the ASIC. The processor may be implemented as a single circuit or implemented by combining a plurality of circuits. The processor implements, as its functions, an acquisition function 311, a generating function 312, a display control function 313, an imaging control function 314, and a system control function 315.

The acquisition function 311 is a function that performs various acquiring operations. The acquisition function 311 acquires, for example, the inner image captured by imaging the inside of the opening OP from the camera C. The inner image is, for example, the optical image captured by the camera C. The acquisition function 311 is an example of an acquisition unit.

The generating function 312 is a function that performs various generating operations. The generating function 312 generates, for example, the visualized image obtained by visualizing the inside of the opening OP viewed from a visual line direction toward the inside of the opening OP based on the inner image acquired by the acquisition function 311. The generating function 312 is an example of a generating unit.

The display control function 313 is a function that performs various display control operations. The display control function 313 causes the monitor M (or the display circuit 34) to display the visualized image generated by the generating function 312. The display control function 313 is an example of a display control unit.

The imaging control function 314 is a function that performs various imaging control operations. The imaging control function 314 controls the high-voltage generator 212, the DAS 214, the rotary drive device 23, the stand drive device 24, and the like so as to cause the gantry 2 to perform X-ray CT imaging in accordance with scan conditions. The imaging control function 314 generates CT image data based on projection data from the DAS 214 and outputs the generated CT image to the monitor M (or the display circuit 34). The imaging control function 314 is an example of an imaging control unit.

The system control function 315 is a function that comprehensively controls each constituent element of the console 3. The system control function 315 controls various functions of the processing circuit 31 based on the input operations accepted from the user via the input circuit 33. The system control function 315 is an example of a system control unit.

The processing circuit 31 implements (1) preprocessing function, (2) reconstruction processing function, and (3) image processing function.
(1) Preprocessing function preprocesses the projection data output from the DAS 214. Preprocessing is, for example, logarithmic conversion, offset correction, sensitivity correction, or beam hardening correction. The preprocessing function is an example of a preprocessing unit.
(2) Reconstruction processing function generates CT image data by performing reconstruction processing of the projection data preprocessed by the preprocessing function. Reconstruction processing is, for example, a filter correction back projection method or a successive approximation reconstruction method. The reconstruction processing function is an example of a reconstruction processing unit.
(3) Image processing function generates predetermined image data by performing image processing (for example, rendering processing) of the CT image data generated by the reconduction processing function. The image processing function may generate predetermined image data based on the input operation accepted from the user via the input circuit 33. The predetermined image data is, for example, the tomographic image data of an arbitrary section or three-dimensional image data. The image processing function is an example of an image processing unit.

The storage circuit 32 is a storage device such as an HDD (Hard Disk Drive), SSD (Solid State Drive), or integrated circuit storage device, which stores various types of information. The storage circuit 32 stores projection data, reconstruction image data, and the like. The storage circuit 32 may be a portable storage medium such as a CD (Compact Disc), DVD (Digital Versatile Disc), or flash memory. The storage circuit 32 may be a drive device that reads and writes various types of information between itself and a semiconductor memory element such as a flash memory or RAM. The save area of the storage circuit 32 may be located inside the X-ray CT apparatus 1 or inside an external storage device connected via a network. The storage circuit 32 may store a database. The storage circuit 32 is an example of a storage unit.

The input circuit 33 accepts various input operations from the user, converts the accepted input operations into electrical signals, and outputs the signals to the processing circuit 31. The input circuit 33 may include a mouse, keyboard, trackball, switches, buttons, joystick, touch pad, and touch panel display. The input circuit 33 may accept an electrical signal from an external input device provided as a separate component from the X-ray CT apparatus 1 and output the accepted electrical signal to the processing circuit 31. The input circuit 33 may be provided on the gantry 2. The input circuit 33 may be a tablet terminal that can wirelessly communicate with the console 3. The input circuit 33 is an example of an input unit.

The display circuit 34 displays various types of information. The display circuit 34 displays the medical image (CT image) generated by the processing circuit 31, a GUI (Graphical User Interface) for accepting various operations from the user, and the like. The display circuit 34 may be an LCD (Liquid Crystal Display), CRT (Cathode Ray Tube) display, OELD (Organic Electro Luminescence Display), plasma display, or the like. The display circuit 34 may be provided on the gantry 2. The display circuit 34 may be a disk top type device, a tablet terminal that can wirelessly communicate with the console 3, or the like. The display circuit 34 is an example of a display unit.

The communication circuit 35 is a circuit that communicates various types of data. The communication circuit 35 communicates CT image data based on the DICOM (Digital Imaging and Communication in Medicine) standard. The console 3 may be connected to a communication network via the communication circuit 35 and connected to an external device via the communication network. The communication circuit 35 is an example of a communication unit.

FIG. 3 is a flowchart showing the operation of the X-ray CT apparatus 1 according to the first embodiment. The X-ray CT apparatus 1 acquires an inner image of the inner space of the opening OP of the scanner 21 and generates and displays a visualized image based on the acquired inner image.
(Step S1A) First of all, the acquisition function 311 acquires the inner image captured by imaging the inside of the opening OP of the scanner 21 from the first visual line direction D1. The acquisition function 311 acquires the inner image captured by the camera C from the camera C.
(Step S2A) The generating function 312 then generates a visualized image based on the inner image acquired in step S1A. The visualized image is the image captured by visualizing the inside of the opening OP when viewed from the second visual line direction D2 defined by an installation position PM of the monitor M as a starting point and the central position PX of the opening OP as an ending point.

In the first embodiment, the first visual line direction D1 coincides with the second visual line direction D2. In this case, the generating function 312 uses the inner image as the visualized image. The generating function 312 may change the magnification percentage of the inner image and use the inner image after the change as the visualized image.

In contrast, if the first visual line direction D1 does not coincide with the second visual line direction D2, the inner image captured when the first visual line direction D1 is viewed from the installation position of the camera C is converted into the visualized image obtained when the second visual line direction D2 is viewed from the installation position PM. For example, the generating function 312 generates the visualized image by viewpoint conversion of the inner image based on the positional relationship between the visual line direction of the first visual line direction Dl and the visual line direction of the second visual line direction D2. A pre-trained machine learning model may be used for viewpoint conversion. A known technique according to image processing may be applied to the generation of a visualized image.
(Step S3A) Finally, the display control function 313 causes the monitor M to display the visualized image generated in step S2A.

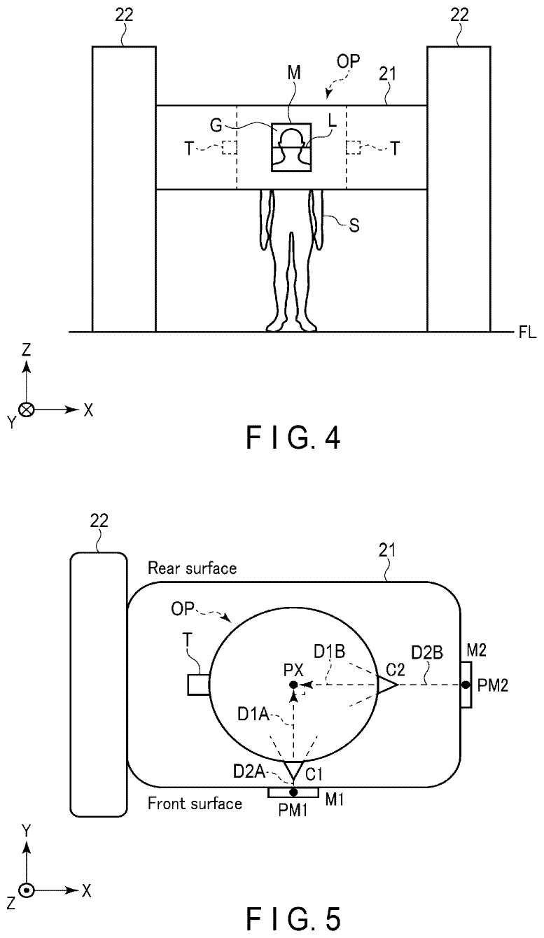

FIG. 4 is an explanatory diagram showing a display example of a visualized image according to the first embodiment. FIG. 4 is a front view when the scanner 21 and the two stands 22 are viewed from the Y-axis negative direction (that is, from the front surface).

A subject S is standing on the floor surface FL (standing position). The head portion and the chest portion of the subject S are inserted in the inner space of the opening OP of the scanner 21. The upper end of the head portion of the subject S is lower than the upper end of the monitor M, and the lower end of the chest portion of the subject S is lower than the lower end of the monitor M. For this reason, the head portion of the subject S and part of the chest portion of the subject S are included in a visualized image G on the monitor M. The visualized image G is displayed when the head portion of the subject S is viewed from the front. Part of the chest portion of the subject S is shielded by the front surface of the scanner 21 and hence is not visually recognized.

The visualized image G may display laser light L projected from the two projectors T onto the subject S. The monitor M is reworded as a transmission window that transmits and displays the inside of the opening OP in a pseudo manner.

According to the first embodiment, the examination technician can check the state of the subject S inside the opening OP of the scanner 21 through the visualized image G displayed on the monitor M. In particular, the examination technician can check the height of an imaging section of the subject S by visually recognizing the height of the laser light L in the visualized image G. Accordingly, the examination technician can intuitively and easily align the height of the imaging section with a desired height in the subject S. The examination technician need not look at the laser light L from below the opening OP and hence experiences less physical load. This enables the X-ray CT apparatus 1 to reduce the physical load associated with imaging of the subject S.

As described above, the gantry 2 is installed in the CT examination room, and the console 3 is installed in the control room adjacent to the CT examination room. The examination technician sometimes checks the subject S in the CT examination room from the control room. In this case, the examination technician can grasp the state of the subject S inside the opening OP of the scanner 21 while taking a perspective of the scanner 21 on the gantry 2.

### Second Embodiment

FIG. 5 is a top view showing the arrangement of a gantry according to the second embodiment. In the second embodiment, one stand 22 grips the left-side surface of a scanner 21 (a cantilever structure).

The scanner 21 may have a plurality of cameras on the inner peripheral side surface forming an opening OP. The plurality of cameras are, for example, a camera C1 and a camera C2. The camera C1 obtains a first optical image (an example of a first inner image) by, for example, imaging the inner space of the opening OP from a first visual line direction D1A. The camera C1 transmits the first inner image to a console 3.

The camera C2 obtains a second optical image (an example of a second inner image) by, for example, imaging the inner space of the opening OP from a first visual line direction D1B. The first visual line direction D1B is, for example, orthogonal to the first visual line direction D1A. The camera C2 transmits the second inner image to the console 3. That is, the two cameras C1 and C2 are installed at different positions and have similar functions. The first inner image and the second inner image are optical images when the inside of the opening OP is viewed from different viewpoints.

A monitor M1 is provided on the front surface of the scanner 21. The monitor M1 displays the first visualized image when the inside of the opening OP is viewed from a second visual line direction D2A. The second visual line direction D2A is the visual line direction defined by an installation position PM1 of the monitor M1 as a starting point and a central position PX of the opening OP as an ending point. The second visual line direction D2A is on the same straight line as the first visual line direction D1A.

A monitor M2 is provided on the right-side surface of the scanner 21. The monitor M2 is installed, for example, at a position that bisects the length of the side surface of the scanner 21 in the long axis direction (Y-axis direction). The monitor M2 displays a second visualized image when the inside of the opening OP is viewed from a second visual line direction D2B. The second visual line direction D2B is the visual line direction defined by an installation position PM2 of the monitor M2 as a starting point and the central position PX of the opening OP as an ending point. The second visual line direction D2B is on the same straight line as the first visual line direction D1B. That is, the two monitors M1 and M2 are installed at different positions and have similar functions.

The two monitors M1 and M2 may be installed in accordance with the direction of a subject. For example, the monitor M1 may be installed so as to face the front surface of the subject. The monitor M2 may be installed so as to face a side surface of the subject. This allows the examination technician to observe the front surface of the subject inside the opening OP by viewing the monitor M1 and to observe a side surface of the subject by viewing the monitor M2. That is, the examination technician can check whether an imaging section is properly aligned in the right and left directions and the front and back directions of the subject.

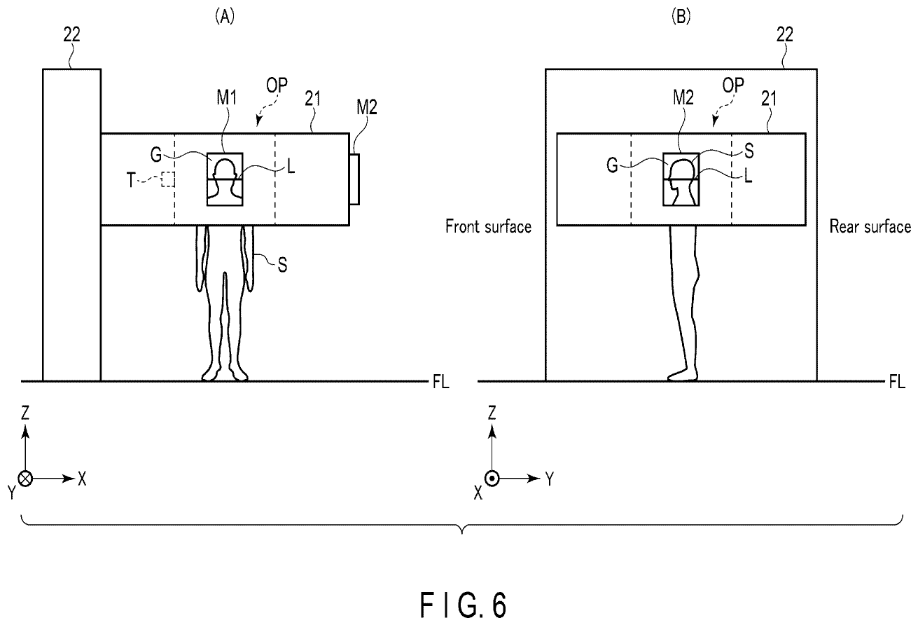

FIG. 6 shows explanatory diagrams showing a display example of a visualized image according to the second embodiment. (A) of FIG. 6 shows the scanner 21 and the stand 22 when viewed from the Y-axis negative direction (that is, from the front surface). (B) of FIG. 6 shows the scanner 21 and the stand 22 when viewed from the X-axis positive direction (that is, from the right-side surface).

As shown in (A) of FIG. 6, a subject S is standing on a floor surface FL. The head portion of the subject S is displayed as a visualized image G on the monitor M1. The subject S and the visualized image G are the same as those shown in FIG. 4. The scanner 21 has the monitor M2 on the right-side surface.

As shown in (B) of FIG. 6, the monitor M2 is installed on the right-side surface of the scanner 21. The monitor M2 is installed so as to face the right-side surface of the subject S. The head portion of the subject S is displayed as the visualized image G on the monitor M2. The visualized image G displays the head portion of the subject S viewed from the right-side surface. Part of the chest portion of the subject S is shielded by the right-side surface of the scanner 21 and hence is not visually recognized. The visualized image G displays laser light L projected on the subject S.

The monitors M1 and M2 each display the visualized image G transmitted through the inside of the scanner 21 in a pseudo manner from a corresponding one of the installation positions (or the viewpoints). The examination technician can observe the head portion of the subject S and the laser light L viewed from the front surface through the visualized image G on the monitor M1. The examination technician can observe the head portion of the subject S and the laser light L viewed from the right-side surface through the visualized image G on the monitor M2.

The monitors M1 and M2 are installed on the scanner 21. When the scanner 21 moves in an arbitrary one of the respective axis directions (the X-axis direction, Y-axis direction, and Z-axis direction), the monitors M1 and M2 also move in a similar direction. That is, the monitor M1 and the monitor M2 need not be moved independently of the scanner 21. The monitor M1 and the monitor M2 each display the visualized image G transmitted through the inside of the scanner 21 in a pseudo manner from the position after the movement. Moving the scanner 21 allows the examination technician to observe the subject S and the laser light L viewed from an arbitrary position after the movement.

In the second embodiment, the camera C1 and the camera C2 may be provided so as to be movable in the circumferential direction of the opening OP. For example, the camera C1 may move so as to be able to always image the front surface of the subject S. The camera C2 may move so as to be able to always image a side surface of the subject S. The examination technician can check the state of the subject S from a specific direction such as the front direction or side-surface direction of the subject S even when the subject S changes its position in the opening OP.

The camera C1 and the camera C2 may differ in type.

For example, the camera C1 and the camera C2 may be an arbitrary combination selected from the group consisting of a visible light camera, a near-infrared light camera, a stereo camera, a depth camera, and the like. The camera C1 and the camera C2 may be made to function as one stereo camera by installing the camera C1 and the camera C2 adjacent to each other.

The camera C1 and the camera C2 may differ in angle of field. For example, the camera C2 may have a wider angle of field than the camera C1. In this case, the camera C1 may image the head portion of the subject S from the front surface, and the camera C2 may image the head portion and the chest portion (or the whole body) of the subject S. The examination technician can check the face of the subject S on the monitor M1 and check a wider region on the monitor M2 than with the camera C1.

### Third Embodiment

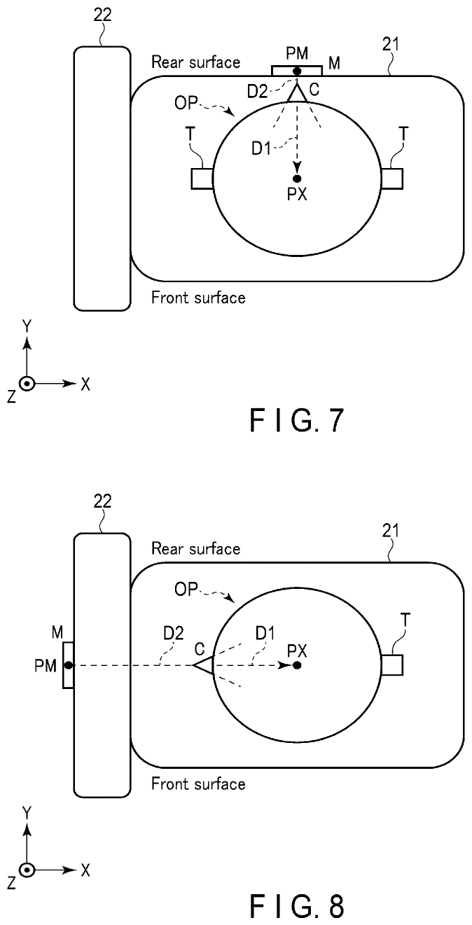

FIG. 7 is a top view showing the arrangement of a gantry 2 according to the third embodiment. As shown in FIG. 7, a monitor M may be installed on the rear surface of a scanner 21. In this case, a first visual line direction D1 of a camera C may be on the same straight line as a second visual line direction D2 of the monitor M. The monitor M displays a visualized image when the inside of an opening OP is viewed from the second visual line direction D2. The visualized image is transmitted through the scanner 21 in a pseudo manner and indicates the inner space of the opening OP.

### Fourth Embodiment

FIG. 8 is a top view showing the arrangement of a gantry 2 according to the fourth embodiment. As shown in FIG. 8, a monitor M may be installed on the left-side surface of a stand 22. That is, the monitor M may be installed on the surface of the stand 22 which is located on the side opposite to the contact surface between a scanner 21 and the stand 22. In this case, a first visual line direction D1 of a camera C1 may be on the same straight line as a second visual line direction D2 of the monitor M. The monitor M displays a visualized image when the inside of the opening OP is viewed from the second visual line direction D2. The visualized image is transmitted through the scanner 21 and the stand 22 in a pseudo manner and indicates the inner space of an opening OP.

### Fifth Embodiment

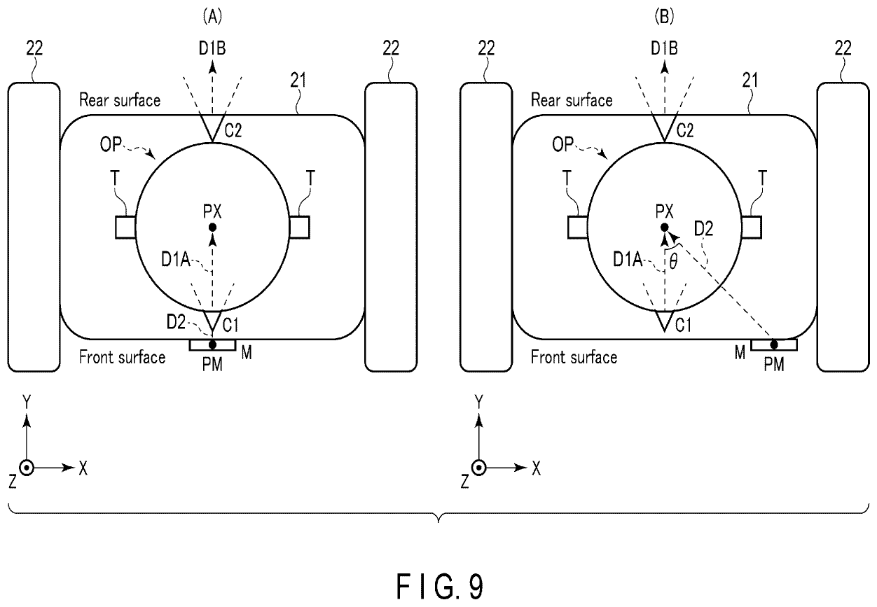

FIG. 9 shows top views showing the arrangement of a gantry 2 according to the fifth embodiment. (A) of FIG. 9 shows a scanner 21 and two stands 22 according to the first arrangement example. (B) of FIG. 9 shows the scanner 21 and the two stands 22 according to the second arrangement example.

As shown in (A) of FIG. 9, the scanner 21 has a camera C1 on a circular inner peripheral surface forming an opening OP. The camera C1 images the inside of the opening OP from a first visual line direction D1A. The camera C1 is similar to the camera C (see FIG. 2).

The scanner 21 has a camera C2 on the rear surface. The camera C2 is installed at a position that bisects the length of the rear surface of the scanner 21 in the long axis direction (X-axis direction). The camera C2 acquires an optical image (rear-surface side image) by imaging the rear surface side of the scanner 21 from a first visual line direction D1B. That is, the camera C2 acquires an optical image (first opposite side image) by imaging the outer space from a side surface (rear surface) facing an installation position PM of a monitor M. The camera C2 transmits the first opposite side image to a console 3. The first visual line direction D1B is on the same straight line as the first visual line direction D1A and a second visual line direction D2.

As shown in (B) of FIG. 9, the second visual line direction D2 of the monitor M sometimes intersects the first visual line direction D1A of the camera C1 at an angle θ. In this case, the first visual line direction D1B is on the same straight line as the first visual line direction D1A but is not on the same straight line as the second visual line direction D2.

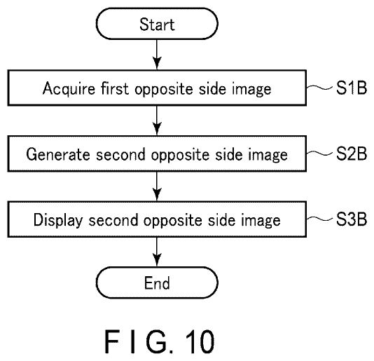

FIG. 10 is a flowchart showing the operation of an X-ray CT apparatus 1 according to the fifth embodiment. An X-ray CT apparatus 1 acquires the first opposite side image by imaging the opposite side of the monitor M to the installation position PM and generates and displays the second opposite side image based on the acquired first opposite side image. The X-ray CT apparatus 1 according to the fifth embodiment may perform an operation similar to that in the first embodiment (see FIG. 3).
(Step S1B) First of all, the X-ray CT apparatus 1 acquires the first opposite side image. More specifically, an acquisition function 311 acquires the first opposite side image captured by imaging the opposite side to the installation position PM of the monitor M. The acquisition function 311 acquires the first opposite side image captured by the camera C2 from the camera C2.
(Step S2B) The X-ray CT apparatus 1 then generates the second opposite side image. More specifically, a generating function 312 generates the second opposite side image based on the first opposite side image acquired in step S1B. The second opposite side image is the first opposite side image viewed from the second visual line direction D2 (viewpoint-converted).

That is, the generating function 312 converts the first opposite side image when the first visual line direction D1B is viewed from the installation position of the camera C2 into the second opposite side image when the second visual line direction D2 is viewed from the installation position PM of the monitor M. This conversion may use a technique similar to that used in step S2A (see FIG. 3). According to the example shown in (A) of FIG. 9, the second opposite side image is similar to the first opposite side image captured by the camera C2. According to the example shown in (B) of FIG. 9, the second opposite side image is an optical image obtained by rotating the viewpoint of the first opposite side image captured by the camera C2 by an angle θ.

(Step S3B) Finally, the X-ray CT apparatus 1 displays the second opposite side image. More specifically, a display control function 313 causes the monitor M to display the second opposite side image generated in step S2B. The display control function 313 may cause the monitor M to display the second opposite side image together with a visualized image G. The display control function 313 may switch and display the visualized image G and the second opposite side image in accordance with the instruction input by the examination technician via an input circuit 33.

According to the fifth embodiment, the examination technician can check the space on the opposite side of the scanner 21 and the stand 22 when viewed from the second visual line direction D2 of the monitor M through the second opposite side image displayed on the monitor M. That is, the examination technician can check the space that is shielded by the scanner 21 and the stand 22 and cannot be visually checked. The examination technician can grasp the existence of an object (for example, an infusion stand and a transportation tool) installed on the opposite side of the scanner 21 and the stand 22. This enables the X-ray CT apparatus 1 to eliminate the load of movement of the examination technician to the opposite side of the scanner 21 and the stand 22.

### Sixth Embodiment

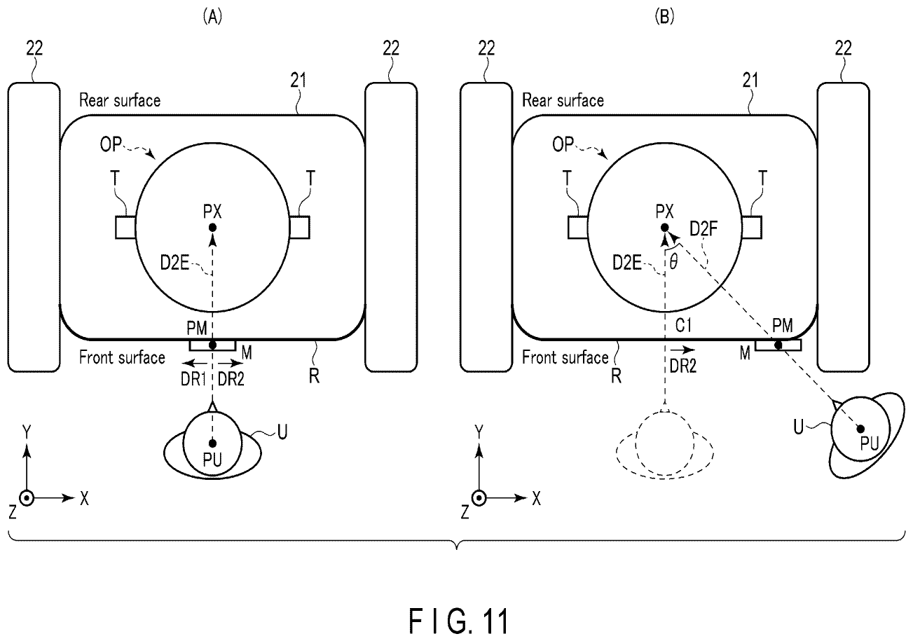

FIG. 11 shows top views showing the arrangement of a gantry 2 according to the sixth embodiment. (A) of FIG. 11 is a top view showing a scanner 21 and two stands 22 according to the first arrangement example. (B) of FIG. 11 is a top view showing the scanner 21 the two stands 22 according to the second arrangement example. For the sake of convenience, a camera C that acquires an inner image is not shown.

As shown in (A) and (B) of FIGS. 11, the scanner 21 has a rail R on the front surface. The rail R extends along the circumferential direction of an opening OP on the front surface of the scanner 21. A moving mechanism (not shown) such as casters engages with the rail R. The moving mechanism moves (or slides) a monitor M along the rail R. The rail R may be installed on the rear surface of the scanner 21 or on the stand 22. The rail R may be installed in an arbitrary direction and path. The rail R may be installed on the surface of at least one of the scanner 21 and the stand 22.

The monitor M moves in a left side direction DR1 or a right side direction DR2 when viewed from a user U (for example, the examination technician) present on the front surface side of the scanner 21. If the user U moves, the monitor M may automatically move following the movement of the user. At this time, the monitor M moves such that the user U is located on a straight line indicating the second visual line direction D2. The monitor M may be manually moved by the examination technician or automatically moved under the control of an X-ray CT apparatus 1 (an imaging control function 314 in particular).

Referring to (A) of FIG. 11, the user U is located in front of the monitor M. A second visual line direction D2E of the monitor M coincides with a straight line passing through a position PU of the user U and a central position PX of the opening OP. Referring to (B) of FIG. 11, the user U moves to the right around the central position PX of the opening OP by an angle e with reference to the second visual line direction D2E. At this time, the monitor M moves in the right side direction DR2 such that a visual line direction D2F of the monitor M coincides with a straight line passing through the position PU of the user U and the central position PX of the opening OP.

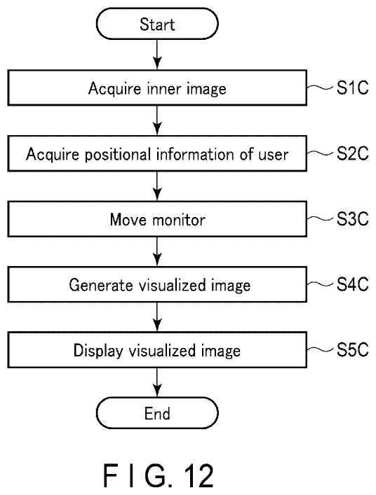

FIG. 12 is a flowchart showing the operation of the X-ray CT apparatus 1 according to the sixth embodiment. The X-ray CT apparatus 1 acquires an inner image of the opening OP of the scanner 21 and acquires the position information of the user U. The X-ray CT apparatus 1 automatically moves the monitor M based on the acquired position information and generates and displays a visualized image G based on an installation position PM of a monitor M after the movement.
(Step S1C) First of all, the X-ray CT apparatus 1 acquires an inner image. Step S1C is similar to step S1A.
(Step S2C) The X-ray CT apparatus 1 then acquires the position information of the user U. More specifically, an acquisition function 311 acquires the position information of the position PU of the user U. The user U may carry a position sensor (for example, a magnetic sensor, ultrasonic sensor, or optical sensor). The position sensor transmits the position information of the user U to a console 3.
(Step S3C) Subsequently, the X-ray CT apparatus 1 moves the monitor M. More specifically, the imaging control function 314 moves the monitor M by driving the moving mechanism of the monitor M based on the position information acquired in step S2C.
(Step S4C) Subsequently, the X-ray CT apparatus 1 generates the visualized image G. More specifically, a generating function 312 generates the visualized image G based on the installation position PM of the monitor M which has moved in step S3C. Step S4C is similar to step S2A.
(Step S5C) Finally, the X-ray CT apparatus 1 displays the visualized image G. Step S5C is similar to step S3A.

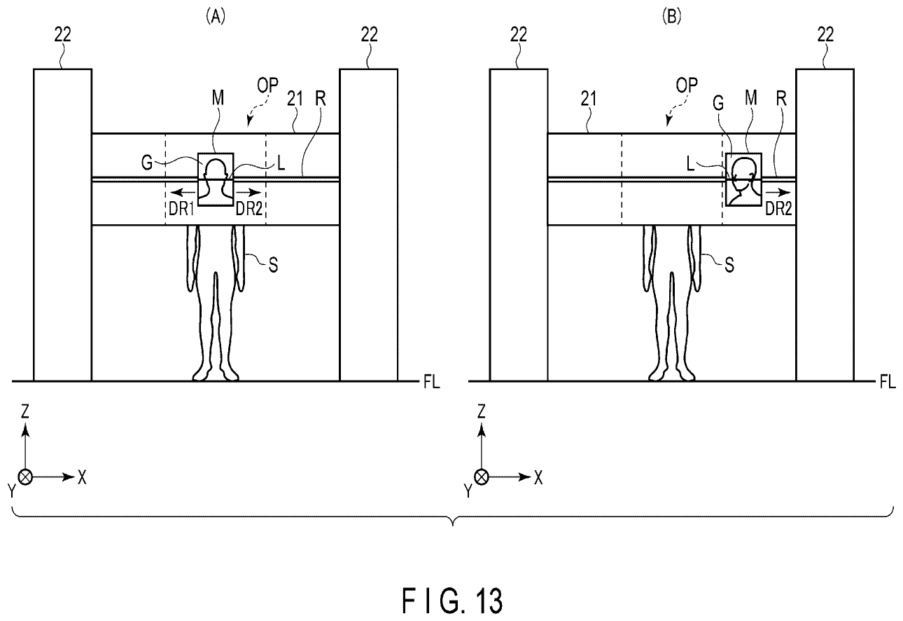

FIG. 13 shows explanatory diagrams showing a display example of the visualized image G according to the sixth embodiment. (A) and (B) of FIGS. 13 are front views showing the scanner 21 and the two stands 22. The state shown in (A) of FIG. 13 corresponds to the state shown in (A) of FIG. 11. The state shown in (B) of FIG. 13 corresponds to the state shown in (B) of FIG. 11.

Referring to (A) of FIG. 13, the rail R extends along a straight line that bisects the height of the front surface of the scanner 21 (the height in the Z-axis direction). The monitor M displays the visualized image G when the head portion of a subject S is viewed from the front direction. Referring to (B) of FIG. 13, the monitor M displays the visualized image G when the head portion of the subject S is viewed from a visual line direction forming the angle θ with the front direction.

According to the sixth embodiment, the examination technician can check the state of the subject S inside the opening OP so as to see through the inside of the opening OP from his/her own position via the monitor M. The monitor M automatically moves on the rail R in accordance with the position of the examination technician. Accordingly, the examination technician can check the subject S from an arbitrary visual line direction and hence can intuitively and easily grasp the spatial position of the laser light L projected on the subject S.

(First Modification) The above embodiment has exemplified the case where the visualized image G displays the actual laser light L projected on the substance S. However, virtual laser light planned to be projected on the substrate S may be displayed on the visualized image G on the monitor M. The visualized image G may not display virtual laser light while the actual laser light L is projected on the substrate S. For example, the display control function 313 can support the examination technician to determine the height of an imaging section when the actual laser light L is not projected because virtual laser light is displayed. The display control function 313 may control display such that virtual laser light is not superimposed on the actual laser light L displayed on the visualized image G. This control allows the examination technician to easily visually check the actual laser light L. In addition, not projecting the laser light L onto the subject S can prevent the subject S from feeling dazzled.

(Second Modification) The visualized image G may display an area finder instead of the linear laser light L. The area finder is a rectangular frame indicating the range (scan area) in which the scanner 21 can perform imaging. The area finder is projected from the projector onto the subject S like the laser light L. As in the first modification, the visualized image G may display a virtual area finder. The visualized image G may not display a virtual area finder while an actual area finder is projected on the subject S. The area finder may be a rectangular frame indicating a range extending from the starting position of X-ray CT imaging by the scanner 21 to the ending position of the imaging (a volume scan range).

(Third Modification) The visualized image G may schematically display the subject S as a schema (pictorial diagram) instead of the actual subject S. The visualized image G may display a schema corresponding to the whole body of the subject S. The visualized image G may display virtual laser light or a virtual area finder superimposed on a schema corresponding to a whole body image. Displaying the schema enables the X-ray CT apparatus 1 to protect the privacy of the subject S.

(Fourth Modification) The visualized image G may be displayed on a monitor different from the monitor M. The monitor M or the different monitor may be an operation panel for the scanner 21, a monitor suspended from the ceiling (a suspended monitor), or the monitor of the console 3 (that is, the display circuit 34). The monitor M or the different monitor may be a portable terminal (for example, a tablet terminal) carried by the user U. The examination technician can precisely observe the visualized image G at a short distance by using his/her own portable terminal.

(Fifth Modification) The visualized image G may be projected onto a surface where the installation position PM of the monitor M is present by using a projector. If, for example, the monitor M is installed on the front surface of the scanner 21, the projector projects the visualized image G onto the whole or part of the front surface of the scanner 21. The projector may project the visualized image G onto the ceiling or a wall surface of the CT examination room. The visualized image G may be projected by using a projection mapping technique. The examination technician can check the projected visualized image G and observe the visualized image G in a range wider than the display screen of the monitor M.

(Sixth Modification) The monitor M may be a liquid crystal display or organic EL display. The monitor M may be installed in accordance with the direction of a subject. For example, the monitor M is installed so as to face the front surface of the subject. The imaging control function 314 may selectively turn on and off the power source of the monitor M in accordance with at least one of (1) an instruction from the examination technician, (2) the posture of the scanner 21, and (3) the imaging mode of the scanner 21. With regard to (1), the examination technician may input the instruction via the input circuit 33. With regard to (2), the posture of the scanner 21 may be a tilt angle around the X-axis or Y-axis. With regard to (3), the respective imaging modes include sets of a plurality of imaging parameters different from each other. The imaging parameters include a tube current, tube voltage, X-ray tube rotating speed, acquisition slice thickness, image reconstruction method, field of view (FOV), reconstruction slice thickness, helical pitch, and interpolation reconstruction method.

(Seventh Modification) The user may move virtual laser light (or a virtual scan area) to a desired position on the visualized image G displayed on the monitor M. The imaging control function 314 may display virtual laser light after the movement in a predetermined display form or may move the scanner 21 in accordance with the position of the laser light. The seventh modification includes steps S1D to S5D described below.

First of all, the user selects virtual laser light on the visualized image G (step S1D). The user selects virtual laser light by a touch operation with a finger, a click operation with the mouse, or the like. The user then moves the selected virtual laser light to a desired position on the visualized image G (step S2D). The user moves the selected virtual laser light by a drag operation or long-press operation with a finger or the mouse.

Subsequently, the imaging control function 314 displays the virtual laser light after the movement on the visualized image G (step S3D). The imaging control function 314 may display the virtual laser light after the movement in a display form different from that of the virtual laser light before the movement. The imaging control function 314 may change the display form (for example, the color, thickness, type (solid line or broken line), and display time (always-on display or blinking display)) of a line indicating the virtual laser light.

Subsequently, the user confirms the position of the virtual laser light after the movement (step S4D). The user confirms the position of the virtual laser light by pressing the confirmation button, speech recognition, or the like. The imaging control function 314 accepts the operation of confirming the position of the virtual laser light by the user.

Finally, the imaging control function 314 moves the scanner 21 in accordance with the position of the virtual laser light after the movement (step S5D). The imaging control function 314 may move the scanner 21 in the vertical direction (Z-axis direction) in accordance with the position. If the X-ray CT apparatus 1 includes a mechanism (for example, a rail) that can move the stand 22 in the horizontal direction (X-axis direction and Y-axis direction), the imaging control function 314 may move the scanner 21 together with the stand 22 by using the mechanism.

According to at least one of the embodiments described above, it is possible to reduce the physical load associated with imaging of a subject. An arbitrary combination of the respective embodiments and the respective modifications can obtain similar effects.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising:
a scanner having an opening into which a subject is inserted;
a stand configured to support the scanner so as to enable the scanner to move in a vertical direction; and
a processing circuit configured to acquire an inner image captured by imaging an inside of the opening from a camera,
generate a visualized image by visualizing the inside of the opening viewed from a visual line direction toward the inside of the opening based on the inner image, and cause a display unit to display the visualized image.

2. The apparatus according to claim 1, wherein the visualized image displays actual laser light that is actually projected from a projector onto the subject and indicates a height of an imaging section in the subject.

3. The apparatus according to claim 1, wherein the visualized image displays virtual laser light that is planned to be projected onto the subject and indicates a height of an imaging section in the subject.

4. The apparatus according to claim 3, wherein the visualized image does not display the virtual laser light while laser light is actually projected from the projector onto the subject.

5. The apparatus according to claim 3, wherein the processing circuit moves a position of the virtual laser light on the visualized image in accordance with an operation from a user and moves the scanner in accordance with the position of the virtual laser light after the movement.

6. The apparatus according to claim 1, wherein the display unit is provided on a surface of at least one of the scanner and the stand, and
the processing circuit acquires an opposite side image captured by imaging a space on an opposite side to the surface provided with the display unit from another camera and causes the display unit to display the opposite side image.

7. The apparatus according to claim 1, further comprising:
a rail provided on a surface of at least one of the scanner and the stand; and
a moving mechanism configured to move the display unit along the rail.

8. The apparatus according to claim 7, wherein the visual line direction is a direction extending from a position where the display unit is provided to a central position of the opening, and
the moving mechanism moves the display unit so as to locate a user on a straight line indicating the visual line direction.

9. The apparatus according to claim 1, wherein the visualized image schematically displays the subject as a schema.

10. The apparatus according to claim 1, wherein the display unit is a portable terminal carried by a user.

11. The apparatus according to claim 1, wherein the display unit is provided on a surface of at least one of the scanner and the stand, and the apparatus further comprises a projector configured to project the visualized image onto the surface provided with the display unit.

12. The apparatus according to claim 1, wherein the display unit is a display, and
the processing circuit selectively turns on and off a power source of the display in accordance with at least one of a posture of the scanner and an imaging mode of the scanner.

13. The apparatus according to claim 1, wherein the display unit is provided on a surface of the stand which is located on an opposite side to a surface that the scanner and the stand face, and
the processing circuit generates a visualized image viewed from a visual line direction extending from a position where the display unit is provided to the central position of the opening.

14. A display method for an X-ray CT apparatus including a scanner having an opening into which a subject is inserted and a stand configured to support the scanner so as to enable the scanner to move in a vertical direction, the method comprising causing a computer to
acquire an inner image captured by imaging an inside of the opening from a camera;
generate a visualized image by visualizing the inside of the opening viewed from a visual line direction toward the inside of the opening based on the inner image; and
cause a display unit to display the visualized image.

15. A non-transitory computer readable medium for an X-ray CT apparatus including a scanner having an opening into which a subject is inserted and a stand configured to support the scanner so as to enable the scanner to move in a vertical direction, the medium including computer executable instructions, wherein the instructions, when executed by a processing circuit, cause the processing circuit to perform a method comprising:
acquiring an inner image captured by imaging an inside of the opening from a camera;
generating a visualized image by visualizing the inside of the opening viewed from a visual line direction toward the inside of the opening based on the inner image; and
causing a display unit to display the visualized image.
